(19) United States
(12) Patent Application Publication
(10) Pub. No.: US 2005/0003524 A1
(43) Pub. Date: Jan. 6, 2005

(54) Module for the culture and/or preservation of microorganisms and the utilization of its metabolic performance

(76) Inventor: Joerg C. Gerlach, Pittsburgh, PA (US)

Correspondence Address: Joerg C. Gerlach MD, PhD, 3613 Butler St, Pittsburgh, PA, 15201, US

(21) Appl. No.: 10/866,280

(22) Filed: Jun. 12, 2004

(30) Foreign Application Priority Data
Jun. 13, 2003 (DE) 103 26 744.1

(51) Int. Cl. A61M 5/00; C12M 1/00

(52) U.S. Cl. 435/293.1

(57) ABSTRACT

This invention is a module used to culture and/or preserve microorganisms, such as mammalian cells or other type of cells, and to utilize their metabolic performance. The module is located in a germ and watertight container and consists of an open open porous material whose pores are in communication with each other. It also consists of at least one tube-like system of hollow pathways whose individual channels intersect and/or overlay each other and communicate with the open porous material. The hollow pathway systems of the invention consist of layers of correlating, parallel running pathways. A hollow pathway system of this bioreactor is constructed from several such layers that are arranged in predetermined spacing on top of each other. With this bioreactor a device is described that facilitates the reorganization and use of microorganisms or cells in a manner typical to that of the natural organ.

This invention is a module used to culture and/or preserve microorganisms, such as mammalian cells or other type of cells, and to utilize their metabolic performance. The module is located in a germ and watertight container and consists of an open porous material whose pores are in communication with each other. It also consists of at least one tube-like system of hollow pathways whose individual channels intersect and/or overlay each other and communicate with the open porous material.

Several devices used for metabolic exchange are already known and used as alternative method in animal research. For the production of organic cells or cell products, especially in the field of liver support systems, devices such as bioreactors, cell perfusion systems, or general modules are currently utilized.

A particularly effective design of such a module is described in EP 059 034 A2 (Gerlach, J. C.)/U.S. Ser. No. 08/117,429: 1993. This patent describes a module designed for the culture and maintenance of microorganisms and the use of its metabolic performance. The module consists of one main chamber that contains at least three independent membrane systems. Of these membrane systems at least two are made of hollow fiber membranes. These hollow fiber membranes form a tightly packed spatial network. The microorganisms are immobilized inside the spaces of the network and/or on the fiber membranes.

Other bioreactors using hollow fiber membranes are presented in WO 00/75275 (McDonald, USA) and EP 1 185 612 (Mc Donald, USA).

Although these bioreactors are capable of supplying fresh substrate to the microorganisms and removing their waste, the hollow fiber membranes can foul after extensive operating time, which can lead to a disruption in function for the microorganisms.

Therefore, the aim of this invention is to develop a superior module for the culture and/or preservation of microorganisms and the use of its metabolic performance, as well as a process for the production, and the use of such a module. This is accomplished by providing a scaffold to immobilize microorganisms and providing hollow pathway systems, eliminating the need for hollow fiber membranes to supply the cells.

The functions of the module have been satisfied by the characteristics of patent claim 1, 28 and 29 and in regards to the process for the production by the characteristics of patent claim 30. The sub claims indicate advantageous continuing development. The utilization is described in claims 42 through 56.

It is suggested that the module consists of a body that is arranged inside a container. The body consists of an open-pore, foam-like open porous structure that exhibits pores, which are able to communicate with each other. Simultaneously this body exhibits at least one channel-like system of hollow pathways whose individual channels intersect and/or overlay each other and perfuse the body to allow the supply and removal of media and cell products via suitable exterior supply and removal structures.

Preferably the module features two independent channel-like systems that intersect and/or overlay each other and infuse the body.

Because the body arranged inside the container is made of open porous material whose pores communicate with each other, connections between the pores via openings in their pore wall to the independent channel-like hollow pathway systems is guaranteed. The microorganisms, in particular the cells, are immobilized within the body, inside the open pores of this foam-like open porous structure. Because of the hollow pathway systems arranged inside this structure, an optimal supply and removal of the substrate carrying nutrients and waste to and from the microorganisms located inside the open pores can occur. Therewith, the bioreactor is a replication of the actual tissue/vascular structure of the natural organs. With this module, for the first time, a bioreactor is available that, within itself, facilitates an optimal perfusion of substrate to supply nutrients and remove waste from microorganisms in every region of the structure in a decentralized array with low gradient while providing a scaffold for the microorganisms.

The hollow pathway systems of the invention consist of layers of correlating, parallel running pathways. A hollow pathway system of this bioreactor is constructed from several such layers that are arranged in predetermined spacing on top of each other. The spacing between the individual channels of the hollow pathway system in one layer and between the individual layers is preferably between 0.5-5 mm. The diameter of the individual channels is preferably 0.1-3 mm.

It is recommended, that the body of the bioreactor feature at least two or more such hollow pathway systems, which cross and/or overlay each other but communicate via the open porous sponge-like structure.

It is preferable that the hollow pathway systems inside the bioreactor be arranged in a crossover design. Therefore, one hollow pathway system consisting of several overlaying layers passes through the body in one direction, and a second hollow pathway system consisting of several overlaying layers passes through the body at another angle, e.g. 30 degrees from the opposite direction. Since the layers are arranged in the spacing described above, the supply and removal of the substrate to the microorganisms, arranged inside the cavities of the open porous structure is guaranteed decentralized with low gradients and high-performance mass exchange in practically every area. Perfusion medium circuits outside the module provide the flow and substrate to support this perfusion. Each circuit infiltrates the body via a system of individual channels and, as a result, metabolic exchange takes place between the channel systems of the open porous structure, between the circuits around the bioreactor, and alongside the cells in the open pores. A circuit that leads to direct perfusion of the microorganisms in the body using counter-current perfusion of two independent channel systems with adequate flow and pressure gradients in each system leads to a high metabolic mass exchange rate, hence the advantage of this design.

The invention includes other arrangements in regards to the geometrical design of the hollow pathway systems and their relationship to other pathways. Therefore, the two hollow pathway systems can cross over at a predetermined angle inside the body. They can also be arranged in parallel on top of each other, which would require more sophisticated flow heads around the open porous sponge-like body.

Because of the high cell density in the bioreactor, sufficient oxygen supply is desired, which can be achieved through high circulation rates of oxygenized media, as well as via oxygen carriers circulating with the media (e.g. synthetic hemoglobin or erythrocytes).

Should the module feature a third independent hollow pathway system, it is advantageous to construct the pathway system in a layer of parallel-arranged hollow pathways. For example, this third hollow pathway permeates the body vertically and interweaves the first two hollow pathway systems.

The invention includes further arrangements in regards to the geometrical design of the third or further hollow pathway systems.

Analogous, a fourth or additional hollow pathway system can be integrated whereby additional functions like cell injection, cell drainage, cell extraction, and movement/flow/pressure application for cell removal are possible.

The first independent hollow pathway system of the bioreactor can be used to supply medium to the microorganisms while the second pathway system would be responsible for supplying oxygen and removing CO₂. The third hollow pathway system assures the removal of the medium. The orientation of the flow between the first and third hollow pathway system may be either counter-current or parallel.

The tube-like hollow pathway systems described above perfuse the open porous body of the module. The pores of the open porous structure are at least the size of a biologic cell and preferably exhibit a diameter of 10-1000 micrometers. It is essential that these pores be inter-connected by hollow spaces (e.g. holes in the wall structure of the pores) to ensure the optimal supply and removal of medium. Thereby, the pores connecting the hollow spaces are preferably 5-500 micrometers in size. This design guarantees that, via the hollow pathway systems, the substrate supplying nutrients can reach any point of the open porous structure and, vice versa, the substrate removing waste from any point of the hollow body can reach their connections to the channels of the hollow pathway system via the pores in the structure. Therefore, the open porous body can also be referred to as an open pore foam or sponge-like structure.

Via the pores the following actions are possible: medium perfusion and exchange, infusion of cells, cell migration, and metabolic exchange or cell product removal.

Oxygen supply can occur via the medium. It can also occur via the hollow pathway systems if additional oxygenation hollow fibers are placed in the lumen. The latter function can also be performed by a fourth hollow pathway system. Another feature of a fourth, or fifth, hollow pathway system can be the support of the removal of growing cells in the body. The latter function can also be supported by balloon-like tubes, which bring movements/forces for cell removal into the body.

With this bioreactor a device is described that facilitates the reorganization of microorganisms or cells in a manner typical to that of the natural organ.

In comparison with afore mentioned inventions, the functional advantages of multi compartment hollow fiber bioreactors are preserved by way of various channel systems in this invention exhibiting the function of various compartments. These channels, however, do not face micro open porous membrane walls, which can foul, and the mechanical stability provided by the hollow fibers in the other systems is replaced by the open porous sponge-like structure of the invention. The disadvantages of potential fouling of the membrane walls are offset using the comparatively open pore body structure.

The open porous body inside the container can exhibit any geometrical shape. It is essential, however, that the open porous structure has an adequate capacity to accommodate a sufficient amount of cells, respectively microorganisms. Therefore it is advantageous that the open porous structure has a volume capacity of 0.5 milliliter-10 liters.

In general, the geometrical form of the body can vary. A block form is, however, preferred because it facilitates the direction of the hollow pathway system from one side to the other and makes it easy to place flow heads on the outer surface.

Only modules with more than three hollow pathway systems require a more complex outer form.

The open porous structure (the body) can be constructed as one single corpus or in a combination of several overlaying, disc or slide-like, single layers that are fixed in the bioreactor.

In regards to the second alternative, the disc/slide-like design, it is advantageous if the disc or slide like individual layers are outfitted with channel shaped ridges. These channel shaped ridges are located on the surface and formed in such a way that, in conjunction with the next preceding layer, a hollow pathway system is formed. Therefore, the ridges are constructed as half channels to form full channels in combination with the next preceding single layer. The advantage of this design is that, from the manufacturing stand point, it is very easy to equip the individual discs/slides with the corresponding ridges. In addition, the individual discs/slides can be developed in such a way that the front wall exhibits another channel-like hollow pathway system in form of infused or drilled channels. Consequently, via the assembly and interconnection of these individual layers, a open porous structure is created that already possesses two independent hollow pathway systems. One hollow pathway system is created through the ridges in the individual layers, whereas the second hollow pathway system is a result of connecting the channels drilled into the individual disks/slides. Further pathways can be placed in the respective free planes.

The open porous body structure, as described above, is located inside a container. The arrangement of the germ- and watertight, sterilizeable container and the open porous body is designed in such a way that the open porous hollow pathways of one system meet in at least one input- and output flow head.

These flow heads for media- or gas perfusion is designed to pass through the container and insure the external supply and removal to and from the body inside the container. They can be separately mounted to the channel-like pathway systems pathway systems. Generally there are two ways to accomplishing this.
1. The inflow/outflow system in itself is part of the container and the connections are implemented by positioning the body inside the container.
2. The inflow/outflow systems have flow heads that are connected to the body.

Preferably, the container features other inlets. One or several inlet serve to flush microorganisms into the module. Other inlets serve to measure pressure, pH, and temperature, insert optical probes for the purpose of microscopy, or to take measurements inside the module using techniques such as fluorescent light. Furthermore, inlets to apply movements/forces for cell harvesting may be used.

The container can be designed in the form of a casing or a foil. The casing design is favored, particularly where injection molding can be used. All known state of the art materials are possible for the manufacturing of the container. It is advantageous with this module that the container and the connections can also be manufactured from reabsorbable/biodegradable material to utilize the module as an implant. Another variation of the casing allows for it to be opened under sterile conditions to remove individual parts of the open porous body occupied by cells, such as individual afore mentioned discs/slides for medical implantation purposes, for molecular biological analysis or for microscopy. For online microscopy, microscopy glass slips may also be incorporated into the housing.

The material for the open porous structure that features previously defined dimensions for the pores and the connections to the pores can be any known state of the art material that results in an open porous foam or sponge-like structure. Once again, as previously mentioned in connection with the container, a biodegradable material can be used.

Preferably, the open porous material consists of sintered ceramic powder like calcium hydroxyapatite. Calcium hydroxyapatite belongs to the group of calcium phosphates that include ceramic materials with varying compositions of calcium and phosphor. It is a compound that exists in nature but can also be manufactured synthetically. Calcium hydroxyapatite is already well known in the medical field as bone replacement material. The motivation for the clinical application of calcium hydroxyapatite is to use a material with similar chemical composition as it occurs in the mineral portion of the bone. Calcium hydroxyapatite makes up 60-70% of the natural mineral component in the bone. Calcium hydroxyapatite powder is generated through the process of precipitation from an aqueous solution by the addition of, for instance, ammonium phosphate to a calcium nitrate solution under alkaline pH conditions. Another preferred material would be aluminum hydroxyapatite.

The powder parts can be connected through a sintering process at temperatures of around 12000 degrees Celsius. Wintermantel describes an example of the manufacture of open porous, solid structures made of calcium hydroxyapatite, e.g. open-pore, foam like structures: the calcium hydroxyapatite powder is mixed with organic additives, that will later on burn away under high temperatures. Thereby it is preferred to use foam-producing substances that become gaseous under increasing temperature and during this process brake open the dividing walls between the foam blisters. (Wintermantel et al: Bio-compatible substances and building elements: implants for medicine and environment. Berlin/Springer 1998: 256-257; ISBN 3-540-64656-6).

An alternative to the use of foam producing substances is to coat the surface of already existing open pore sponge like structures, e.g. synthetic or natural sponges, or scaffolds which structures evaporate during the sintering process.

Another alternative exists in the application of a hollow fiber membrane module according to EP 059 034 A2, and U.S. Ser. No. 08/117,429: 1993 (Gerlach J. C.) that is, filled with afore mentioned open porous foam builder, or ceramic suspension prior to the sintering process. The module described above is generally suitable for the culture and/or maintenance of any kind of microorganisms, especially for cells and mammalian cells including: cell lines, immortalized cells, stem cells, organ cells and/or co-cultures of various cells.

The invention also pertains to preferred manufacturing processes of afore described module.

The first step is to create a open porous sponge-like structure whose pores can communicate with each other and has at least one independent channel-like hollow pathway system whose hollow pathways pervade the body. In the second step this structure is placed inside a sterilizeable and watertight container.

In regards to afore described open porous structure, the body of the module can be manufactured either as one single piece or through interconnection of a disc/slide like arrangement.

In the event that a body composed of single layer open porous structures is manufactured, as initially described, e.g. via a frothing and sintering process with, for example, calcium hydroxyapatite, hole-like hollow pathways are infused/drilled into the open porous structure in a second step. Generally any manufacturing process that can implement hollow pathways is suitable, such as laser technology, drilling, milling, or the use of molds already exhibiting such pathways, while the body is cast.

A second alternative to manufacturing the open porous structure is to fabricate single layers made of a material such as calcium hydroxyapatite and outfit the surface of the individual layers with channel-like ridges. Equipping the surface of the disc or slide shaped single layers with channel-like ridges can take place during the production of the single layers by using an appropriate shaping process, or the use of molds already exhibiting such pathways, while the disc/slide is cast.

These disc/slide shaped single layers, e.g. can then be outfitted with the second independent tube-like system using laser cutting or drilling processes on the front wall.

Alternatively, open pore, sponge like structures can be coated with a material such as a calcium hydroxyapatite suspension and the channels applied before the sintering or coating process.

In the case of using layered discs or slides for the body, the interconnection of the single layers has to be guaranteed. This can be achieved by clamping them into a separate fixture or the container developed as a case that holds the discs or slides together. This second option offers procedural advantages.

Afore described open porous sponge-like structure will then be placed in the container, preferably into a container made from injection molding. Afore mentioned structure can also be coated with a housing/container forming material or placed into a foil-like housing. In regards to the inflow- and outflow it has to be merely assured that there is at least one input- and output system available per independent hollow pathway system.

The further steps serve the preparation of cells and their culture in the open pore foam like structures of the module. Hereunto, cleaning with an agent such as aqueous media can take place. Typically, sterilization should occur.

Coating of the open porous material in the module with biomatrix, or scaffolds, using materials such as collagen is a state of the art procedure. When using biomatrix-producing cells in co-culture in the body, a coating with foreign biomatrix is preventable. After parenchymal and non-parenchymal cells are infused, an organ-typical culture can begin in the body.

Some organs produce cells that are later flushed from the tissue via the bloodstream, e.g. bone marrow stem cells. Similarly, the flushing of produced cells such as immune cells, blood cells, and/or stem cells out of the module can lead to cell harvest.

The harvesting of cells can occur by flushing the open porous structure with culture media, possibly after enzymatic digestion of the biomatrix with collagenase/trypsin. The harvesting may also be supported by structures, providing movements/forces to the cell. Generally, the module is suitable for the maintenance, preservation, reproduction, and/or utilization of individual cells, various kinds of cells (co-cultures), cell lines, or immortalized cells, or stem cells of an organ or of several organs. The utilization of the bioreactor can be used in the industrial production of diagnostic or therapeutic substances through cells, or in the production of cells for industrial use as well as for therapeutic transplantation, as well as in basic or industrial research.

The module can also be used to offer cell performance to a patient in form of an extra-corporeal hybrid organ. In addition, the module can be applied to the development of implantable organs transplants. The module is also suitable as an alternative laboratory system to supplement animal experiments in research and pharmacology. It can also be utilized to create cell systems for the multiplication or reproduction of viruses, such as HIV and hepatitis B/C viruses. The module can also serve for the production of vaccines. The device is especially suitable for the reorganization, maintenance and preservation of stem cells as well as their growth and differentiation to organ tissues.

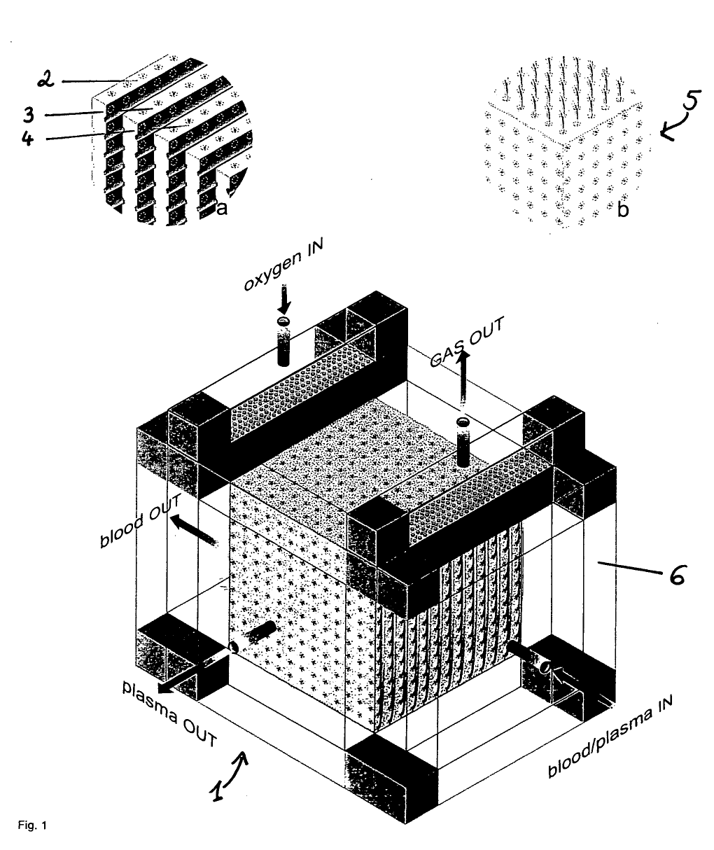

The invention is described as follows in FIGS. 1-4.

FIG. 1 describes the schematic structure of the module.

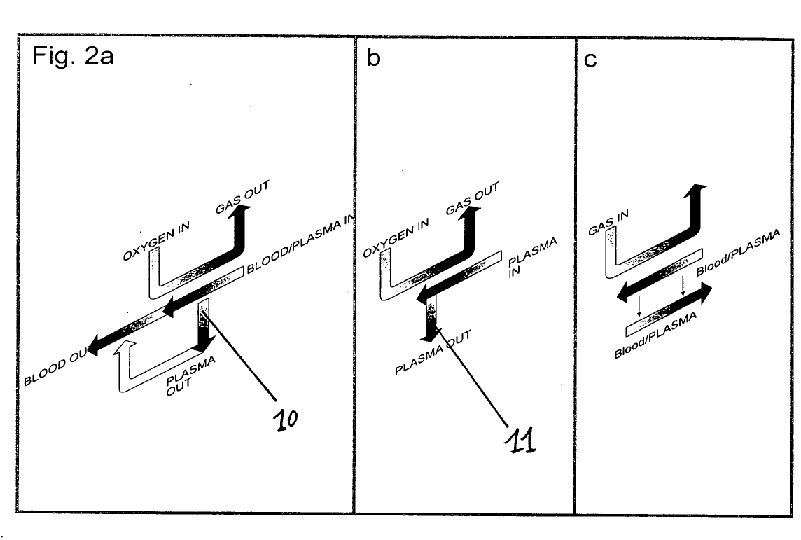

FIG. 2 describes schematically various possibilities for process management.

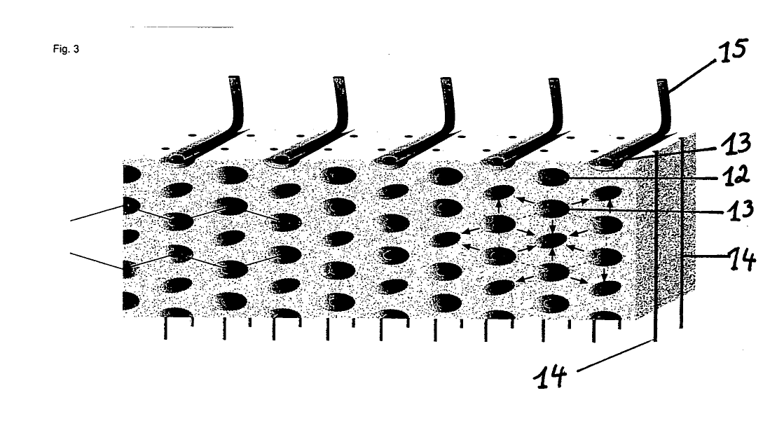

FIG. 3 describes another variation of the invention using additional hollow fiber capillary membranes.

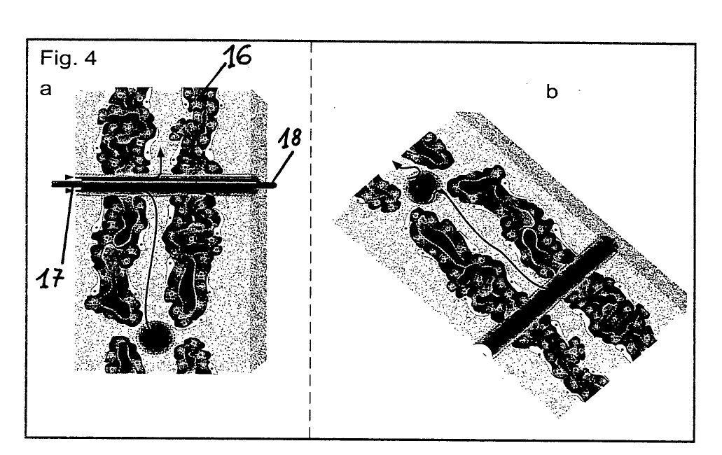

FIG. 4 depicts how the cells are immobilized in the module.

FIG. 1 describes the schematic structure of module 1 where the open open porous structure exists in the form of a cuboid block.

The cuboid block can be constructed, as shown in FIG. 1a, from single discs 2, 3, 4, or from one uniform block as shown in FIG. 1b. As shown in FIG. 1, block 5 is constructed from open porous ceramic and exhibits three independent hollow pathway systems. The hollow pathway systems, as shown in FIG. 1, are generated from individual layers, meaning the respective individual layers consist of single parallel channels. Two hollow pathway systems exist in one plane and cross each other at a 90-degree angle. The hollow pathways are overlaying each other. The third hollow pathway system passes through the open porous structure vertically from top to bottom and intertwines the first two hollow pathway systems. One of the systems is threaded with oxygenating hollow fiber membranes 15. Alternatively the perfusion with oxygenated substances, like hemoglobin or erythrocytes, is possible. The first hollow pathway system is therefore responsible for oxygen inflow and outflow via oxygenating membrane 15. The second hollow pathway system is responsible for the outflow of media and the third, running perpendicular to the first two, is responsible for media outflow. The last two pathway systems with media can be used in either parallel or counter current process perfusion. The hollow pathway systems are arranged in such a way the conditions for metabolic exchange inside the module is identical at any point.

The module, as shown in FIG. 1, has as an outer housing 6 that is preferably generated by injection molding. The appropriate inlets and outlets are an integral part of the housing.

Examples for the process management of the independent hollow pathway systems are described in FIG. 2a-c. The arrows symbolize the direction of media flow and are not synonymous with the hollow pathway systems. FIG. 2a describes the utilization of only one hollow pathway system that is threaded with an oxygenating hollow fiber membrane. One hollow pathway system is used for the infusion of media, respectively blood, and metabolic exchange occurs to and from the pores via diffusion. The downward pointing arrow (10) describes the diffusion direction of the media, respectively blood plasma. In addition, an oxygenating hollow fiber is threaded into the hollow pathway system (not shown), which analogous to FIGS. 1 and 3 allows for a decentralized oxygen supply inside the hollow pathway system. The arrows in FIG. 2b illustrate oxygen exchange across two independent hollow pathway systems (not shown), where the first one serves for media inflow and the second one serves for media outflow and metabolic exchange between the media and the microorganism occurs in the open pores via perfusion. The downward pointing arrow (11) depicts the media transport into the second hollow pathway system. Analogous to FIG. 2a, an additional oxygenating hollow fiber is threaded into the first hollow pathway system (not shown), which allows for a decentralized oxygen supply inside the media admitting hollow pathway system.

FIG. 2c corresponds with FIG. 2b. Two independent hollow pathway systems are however arranged in parallel and at the same angle. The media is supplied via counter current perfusion between the two independent hollow pathway systems, whereby, with increased flow rate an improved metabolic exchange is possible.

FIG. 3 describes another version of the invention depicting three independent hollow pathway systems. Characteristic for this version of the invention is that an oxygenating hollow fiber 15 is threaded into the first of the three independent hollow pathway systems 12, 13, 14. This arrangement can be advantageous when it becomes necessary to perform decentralized inflow of oxygen through one of the hollow pathways and equal distribution of oxygen has to be guaranteed. To achieve this an oxygenating hollow fiber is threaded into hollow pathway system that is dimensioned and specifically constructed to be permeable for oxygen.

As a result, the decentralized, even, and homogenous distribution of oxygen in the hollow structure is guaranteed.

Analogous to FIG. 2b metabolic exchange via perfusion occurs between the first independent system 12 and the second independent system 13 (symbolized by arrows). Additionally, a third independent hollow pathway system (14) is described which could be used for cell drainage or, should it be used with liver cells, as well as for biliary drainage.

FIG. 3 describes an arrangement where it can be used advantageously with liver cell cultures because the physiological situation of liver arteries (analogous to the function of membrane 15), portal veins (analogous 12), liver veins (analogous 13), and biliary ducts (analogous 14) is imitated.

FIG. 4 schematically describes in FIGS. 4a and 4b how the cells immobilize inside the module. FIG. 4 only shows a sectional segment of the open porous hollow structure. In the open pores between the hollow pathway systems cells 16 are immobilized. They are admitted in with media via one hollow pathway system analogous to FIG. 2b. The media comes in contact with the cells through the open pore structure, exchanges nutrients and waste and leaves through the second hollow pathway system. In an example, an oxygenating hollow fiber 18 (respectively 15 FIG. 3) is integrated into the first system 17 (respectively 13 FIG. 3) analogous to FIG. 2 to supply the cells with oxygen. The arrows describe the flow direction for the media from the first independent hollow pathway system via the open pores to the second system. FIG. 4 describes a section of the structure in the plane parallel to the oxygenating hollow fibers. FIG. 4b describes the same structure in a viewing plane perpendicular to FIG. 4a, showing flow of gas from the oxygenating hollow fiber.

1. Module for the culture and use of metabolic performance and/or maintenance of microorganisms, particularly cells, arranged in a germ- and watertight structure, consisting of an open porous material, whose pores are in communication with each other and has at least one communicating tube-like hollow pathway system whose individual hollow pathways cross each other and/or overlay each other and infuse the body.

2. Module according to patent claim 1, is characterized by at least two independent tube like hollow pathway systems.

3. Module according to patent claim 2, is characterized by a tube like hollow pathway system that consists of at least one plane of parallel to each other running, individual channels.

4. Module according to patent claims 2 and 3 is characterized by a hollow pathway system that consists of multiple overlaying planes each with parallel individual channels.

5. Module according to at least one of the patent claims 24, characterized by the existence of three independent tube like hollow pathway systems.

6. Module according to at least one of the patent claims 2-5, characterized by the presence of four or more independent hollow pathway systems.

7. Module according to at least one of the patent claims 2-6, thereby characterized that each channel in the hollow pathway system has a diameter of 0.1-3 mm.

8. Module according to at least one of the patent claims 3-7, thereby characterized that the distance between each individual, parallel to each other running channels of a hollow pathway system arranged in one plane and/or in between the planes is 0.5-5 mm.

9. Module according to at least one of the patent claims 1-8, thereby characterized that the open pores of the porous structure have a diameter of 10-1000 micrometer.

10. Module according to at least one of the patent claims 1-9, thereby characterized that the pores are connected to each other through pore wall openings 10-500 micrometer in size.

11. Module according to at least one of the patent claims 1-10, thereby characterized that the structure provides a cell compartment volume of 0.5 ml-10 liters.

12. Module according to at least one of the patent claims 1-11, characterized by a block shaped structure

13. Module according to at least one of the patent claims 1-12, thereby characterized that the core structure consists of one piece.

14. Module according to at least one of the patent claims 1-12, thereby characterized that the core structure is a network of multiple, overlaying, slide- or disc shaped, single layers held together by the container.

15. Module according to at least one of the patent claims 1-14, thereby characterized that at least one surface of the disc shaped single layers exhibit a pattern of ridges that are arranged and dimensioned in such a way that by interconnection with the next single layer a tube like hollow pathway system is formed.

16. Module according to at least one of the patent claims 14-15, thereby characterized that, from the front to the back wall, the disc shaped single layers are infused by a further tube like hollow pathway system.

17. Module according to patent claims 14 through 16, thereby characterized that, from another surface to the opposite surface, the disc/slide like shaped individual layers are infused with hollow pathways.

18. Module according to at least one of the patent claims 1-17, thereby characterized that each tube like hollow pathway of a system ends in at least one inflow/outflow head.

19. Module according to patent claim 18, thereby characterized that the inflow and outflow systems are connected to the porous structure.

20. Module according to patent claim 18, thereby characterized that the inflow and outflow system is an integral part of the container.

21. Module according to at least one of the patent claims 1-20, thereby characterized that the porous structure consists of biodegradable material.

22. Module according to patent claim 21, thereby characterized that the walls of the open porous material are generated from sintered ceramic powder.

23. Module according to patent claim 22, thereby characterized that a) the open porous structure is made of a frothed suspension containing ceramic powder and additives and the pores partially brake open during the sintering process; b) or the structure was generated from frothed suspension containing ceramic powder and additives which was, prior to the sintering process, foamed between a capillary membrane bioreactor structure; c) or the open porous body is generated from an open porous, sponge like, basic structure from a suspension containing ceramic powder and additives, which burnt during the subsequent sintering process; d) or the pores structure is directly constructed from this sponge like basic structure.

24. Module according to patent claim 1-23, thereby characterized that the container is a case.

25. Module according to patent claim 24, thereby characterized that the container is made through injection molding.

26. Module according to patent claim 1-25, thereby characterized that the container is a foil.

27. Module according to patent claim 1-26, thereby characterized that the container consists of biodegradable material.

28. Module according to patent claim 1-27, thereby characterized that cell lines, stem cells, organ cells and/or co-cultures of various cells are immobilized inside the open porous structure.

29. Module according to patent claim 1-28, thereby characterized that hollow fiber membranes for oxygenation, dialysis, heat exchange and/or other technical functions such as a separate co-culture, or cell removal features, are built into at least one hollow channel-like hollow pathway system.

30. Procedure to manufacture the module according to at least one of the patent claims 1-29 with the following steps:
Manufacturing of a open porous structure whose pores are in communication with each other and that is infused by at least one independent channel like hollow pathway system.
Inserting the structure in a germ- and watertight, sterilizeable container
Sterilization
Cell seeding
Cell culture

31. Procedure according to patent claim 30, thereby characterized that a one-piece open porous structure is generated and, in a second step, channel like hollow pathways are drilled into this open porous structure.

32. Procedure according to patent claim 31, thereby characterized that the channel-like hollow pathways are drilled via drill, laser, and/or bur.

33. Procedure according to patent claim 32, thereby characterized that hollow fiber membranes, such as oxygenation membranes, are threaded into at least one hollow pathway.

34. Procedure according to patent claims 30 through 33, thereby characterized that the open porous structure is made of a network of disk/slide like single layers.

35. Procedure according to patent claim 34, thereby characterized that the surfaces of the disc/slide like layers are outfitted with channel like ridges.

36. Procedure according to patent claim 35, thereby characterized that the front walls of the disc/slide like single layers are outfitted with channel like hollow pathway systems, and hollow pathways are drilled into the surfaces from one side to the opposite side.

37. Procedure according to patent claim 36, thereby characterized that the hollow pathways are drilled via drill, laser, and/or bur.

38. Procedure according to patent claim 34-37, thereby characterized that prior to assembly, hollow fiber membranes, such as oxygenation membranes, are threaded into one of the hollow pathway systems.

39. Procedure according to patent claim 30-38, thereby characterized that the open porous structure consists of biodegradable material.

40. Procedure according to patent claim 39, thereby characterized that the open porous structure is generated via sintering process from frothed ceramic powder and additives; and that during the sintering process the pores partially brake open; or the structure was generated from frothed suspension containing ceramic powder and additives which was, prior to the sintering process foamed between a capillary membrane bioreactor structure; c) or the open porous body is generated from an open porous, sponge like, basic structure from a suspension containing ceramic powder and additives, which burnt during the subsequent sintering process; d) or the pores structure is directly constructed from this sponge like basic structure.

41. Procedure according to patent claim 40, thereby characterized that calcium- or aluminum hydroxyapatite is used as basic ceramic substance.

42. Utilization of the module according to at least one of the claims 1 through 29 for the culture, maintenance, differentiation, proliferation, and/or utilization of individual cells and/or different cells (co-cultures) of an organ.

43. Utilization according to patent claim 42 for stem cells, including embryonic stem cells.

44. Utilization according to patent claim 42 for human stem cells.

45. Utilization according to patent claim 42 for adult stem cells.

46. Utilization according to patent claim 42 for the production of cells.

47. Utilization according to patent claim 42 for the production of progenitor cells for cell transplantation, or culture in other culture systems

48. Utilization according to patent claim 42 for the modification/production of gene technological modified cells, immortal cells, cell lines, and/or trans-genetic cells.

49. Utilization of the module according to one of the patent claims 1-29 for the production of substances by cells.

50. Utilization of the module according to one of the patent claims 1-29 for the differentiation of organ typical cells from stem cells.

51. Utilization of the module according to one of the patent claims 1-29 for the generation of organ typical structures from adult stem cells, fetal and/or embryonic cells.

52. Utilization of the module according to one of the patent claims 1-29 as extracorporeal hybrid organ for organ support, e.g. hybrid liver or hybrid lung.

53. Utilization of the module or part thereof according to one of the patent claims 1-29 as implantable cell/organ transplants.

54. Utilization of the module according to one of the patent claims 1-29 as system to replace and/or supplement research with animals.

55. Utilization of the module according to one of the patent claims 1-29 as in vitro virus culture and virus reproduction system.

56. Utilization according to one of the afore mentioned claims for the production of cellular substances, e.g. cellular metabolic products, known or unknown mediators, hormones, differentiation factors, signal molecules, growth factors, sensitization factors, cytokines, proteins, anti bodies, vaccines, and/or the production of organ specific biomatrix substances.

57. Utilization according to at least one of afore mentioned claims for the development of a hybrid gland.

58. Utilization according to at least one of the afore mentioned claims for the production of organic cells like stem cells or differentiated cells of a specific organ, blood cells, immune system cells.

59. Utilization according to at least one of afore mentioned claims as hybrid immune system for the production of immune competent cells and vaccines, progenitor cells for organs and blood components.

60. Utilization according to at least one of afore mentioned claims as hybrid blood cell system (hybrid bone marrow) for the production of blood components especially blood platelets, erythrocytes and white blood cells.

61. Utilization according to at least one of afore mentioned claims as hybrid stem cell system for the production of progenitor cells for organs, especially for the transplantation of repair cells.

62. Utilization according to at least one of afore mentioned claims in cell based therapy, regenerative medicine, cell biology and/or the development of and production of vaccines.
